(19) United States
(12) Patent Application Publication
(10) Pub. No.: US 2007/0128233 A1
(43) Pub. Date: Jun. 7, 2007

(54) Cosmetic composition having a unique cushiony texture

(75) Inventors: Shao Xiang Lu, Kang Qiao Town (CN); Wei Hong Yu, Edison, NJ (US)

Correspondence Address: L'OREAL USA/ PATENT DEPARTMENT, 30 TERMINAL AVENUE, CLARK, NJ, 07066, US

(73) Assignee: L'OREAL, Paris (FR)

(21) Appl. No.: 11/602,154

(22) Filed: Nov. 20, 2006

Related U.S. Application Data
(60) Provisional application No. 60/742,400, filed on Dec. 5, 2005.

(51) Int. Cl. A61K 8/02; C08K 5/5415

(52) U.S. Cl. 424/401; 524/261

(57) ABSTRACT

A method of making-up a keratinous substrate comprising contacting the keratinous substrate with a cosmetic composition containing: (a) at least one polyorganosiloxane-containing polymer having at least one moiety containing: (i) from 1 to about 1000 organosiloxane units; and (ii) at least two groups capable of establishing hydrogen interactions; (b) an oil phase containing at least one oil component; and (c) at least one cured silicone powder coated with microfine particles.

This application is a non-provisional application of, and claims benefit to, U.S. Provisional Application No. 60/742,400, filed Dec. 05, 2005, which is incorporated by reference in its entirety for all purposes.

### BACKGROUND OF THE INVENTION

Many cosmetic compositions, including pigmented cosmetics such as foundations, concealers, lipsticks, mascaras, and other cosmetic and sunscreen compositions, have been developed for comfortable application and wear. However many of these compositions are difficult to apply and do not have a smooth feel upon application. Furthermore, compositions may have a tendency to be tacky, resulting in poor application and spreadability characteristics. There is still a need, therefore, for improved long-wearing cosmetic compositions with cushiony, soft and silky feel upon application.

Accordingly, a need exists for a cosmetic composition which is stable, with good pay off, spreadability and unique texture resulting in an excellent sensory feel.

### SUMMARY OF THE INVENTION

The present invention is directed to a method of making-up a keratinous substrate involving contacting the keratinous substrate with a cosmetic composition containing: (i) at least one polyorganosiloxane-containing polymer, (ii) an oil phase containing at least one oil component, and (iii) at least one cured silicone powder coated with microfine particles.

### DETAILED DESCRIPTION

Other than in the operating examples, or where otherwise indicated, all numbers expressing quantities of ingredients and/or reaction conditions are to be understood as being modified in all instances by the term “about”.

It has been surprisingly discovered that by combining at least one polyorganosiloxane-containing polymer, an oil phase containing at least one oil component, and at least one cured silicone powder coated with microfine particles, a (semi)solid cosmetic composition can be formed having a unique texture with a powdery yet cushiony feel, good pay-off and excellent storage stability.

The Polyorganosiloxane-containing Polymer

The polyorganosiloxane-containing polymer useful herein is a polymer (homopolymer or copolymer) having at least one moiety which contains: at least one polyorganosiloxane group consisting of 1 to about 1000 organosiloxane units in the chain of the moiety or in the form of a graft, and at least two groups capable of establishing hydrogen interactions.

The polyorganosiloxane-containing polymers may comprise at least one moiety corresponding to formula (I):

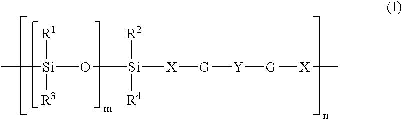

in which:

1) R¹, R², R³ and R⁴, which may be identical or different, represent a group chosen from:
(a) linear, branched or cyclic, saturated or unsaturated, C₁ to C₄₀ hydrocarbon-based groups, possibly containing in their chain one or more oxygen, sulphur and/or nitrogen atoms, and possibly being partially or totally substituted with fluorine atoms,
(b) C₆ to C₁₀ aryl groups, optionally substituted with one or more C₁ to C₄ alkyl groups,
(c) polyorganosiloxane chains possibly containing one or more oxygen, sulphur and/or nitrogen atoms;

2) X, which may be identical or different, represents a linear or branched C₁ to C₃₀ alkylenediyl group, possibly containing in its chain one or more oxygen and/or nitrogen atoms;

3) Y is a saturated or unsaturated, C₁ to C₅₀ linear or branched divalent alkylene, arylene, cycloalkylene, alkylarylene or arylalkylene group, optionally comprising one or more oxygen, sulphur and/or nitrogen atoms, and/or optionally substituted with one of the following atoms or groups of atoms: fluorine, hydroxyl, C₃ to C₈ cycloalkyl, C₁ to C₄₀ alkyl, C₅ to C₁₀ aryl, phenyl optionally substituted with one to three C₁ to C₃ alkyl, C₁ to C₃ hydroxyalkyl, and C₁ to C₆ aminoalkyl groups;

4) G, which may be identical or different, represents a group chosen from ester, amide, sulphonamide, carbamate, thiocarbamate, urea, thiourea groups, and combinations thereof;

5) m is an integer ranging from 1 to 1,000, preferably from 1 to 700 and more preferably from 6 to 200; and

6) n is an integer ranging from 2 to 500 and preferably from 2 to 200.

The polyorganosiloxane-containing polymers may also comprise at least one moiety corresponding to formula (II):

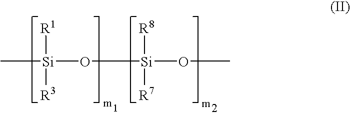

in which
R¹ and R³, which may be identical or different, are as defined above for formula (I),
R⁷ represents a group as defined above for R¹ and R³, or represents a group of formula —X-G-R⁹ in which X and G are as defined above for formula (I) and R⁹ represents a hydrogen atom or a linear, branched or cyclic, saturated or unsaturated, C₁ to C₅₀ hydrocarbon-based group optionally comprising in its chain one or more atoms chosen from O, S and N, optionally substituted with one or more fluorine atoms and/or one or more hydroxyl groups, or a phenyl group optionally substituted with one or more C₁ to C₄ alkyl groups,
R⁸ represents a group of formula —X-G-R⁹ in which X, G and R⁹ are as defined above,
m₁ is an integer ranging from 1 to 998, and
m₂ is an integer ranging from 2 to 500.

According to another embodiment, it is also possible to use a copolymer comprising several different moieties of formula (I), and/or several different moieties of formula (II), that is to say a polymer in which at least one of the groups R¹, R², R³, R⁴, X, G, Y, m and n is different in one of the moieties.

It is also possible to use a copolymer comprising at least one moiety of formula (I) and at least one moiety of formula (II), the moieties of formula (I) and the moieties of formula (II) possibly being identical to, or different from, each other. These copolymers may be block copolymers or grafted copolymers.

Additional polyorganosiloxane-containing polymers which may be used in the composition of the invention include those described in documents U.S. Pat. No. 5,874,069, U.S. Pat. No. 5,919,441, U.S. Pat. No. 6,051,216, and U.S. Pat. No. 5,981,680, the entire contents of which are hereby incorporated by reference.

A preferred polyorganosiloxane-containing polymer for use in the present invention will have at least one moiety chosen from formula (III):

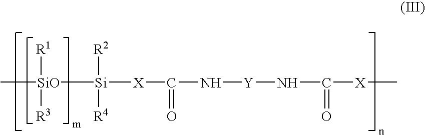

and formula (IV)

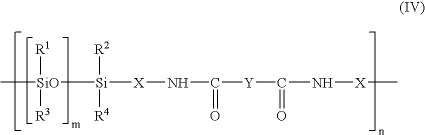

in which:

(a) R¹, R², R³ and R⁴ are the same or different and may be selected from the group consisting of methyl, ethyl, propyl, isopropyl, a siloxane chain, and phenyl;

(b) X is a linear or branched chain alkylene having 1-30 carbons;

(c) Y is selected from the group consisting of linear or branched chain alkylenes having 1-40 carbons;

(d) m is an integer ranging from 1 to 700; and

(e) n is an integer ranging from 1 to 500.

Particularly preferred polyorganosiloxane-containing polymers useful herein are commercially available from Dow Corning as DC 8178 and DC 8179, which are known under the INCI denomination of Nylon-611/Dimethicone Copolymer.

The polyorganosiloxane-containing polymer can be present in the composition according to the invention in an amount of from about 0.1 to about 50% by weight; such as from about 0.5 to about 40% by weight; such as from about 2 to about 30% by weight; such as from about 2 to about 20% by weight; such as from about 2 to about 15% by weight, all weights being based on the total weight of the composition.

Oil Phase

The composition of the invention also contains an oil phase having at least one oil component, which may be volatile or non-volatile, chosen from hydrocarbon-based oils, silicone oils and mixtures thereof.

The expression “volatile oil” means any non-aqueous medium capable of evaporating on contact with the skin or the lips in less than one hour at room temperature and atmospheric pressure. The volatile oil in the composition of the invention is an organic oil, liquid at room temperature, having a non-zero vapor pressure, at room temperature and atmospheric pressure, ranging in particular from 10⁻² to 300 mmHg (1.33 to 40,000 Pa) and, for example, greater than 0.03 mmHg (4 Pa) and further example greater than 0.3 mmHg (40 Pa).

The expression “non-volatile oil” means any non-aqueous medium capable of remaining on the skin or the lips at room temperature and atmospheric pressure for at least several hours, such as those having a vapor pressure of less than 10⁻² mmHg (1.33 Pa).

The expression “hydrocarbon-based oil” means an oil comprised of carbon and hydrogen atoms, optionally having at least one group chosen from hydroxyl, ester, carboxyl and ether groups.

Examples of suitable volatile hydrocarbon-based oils include, but are not limited to, volatile hydrocarbon oils having from 8 to 16 carbon atoms and their mixtures and in particular branched C₈ to C₁₆ alkanes such as C₈ to C₁₆ isoalkanes (also known as isoparaffins), isododecane, isodecane, isohexadecane, and for example, the oils sold under the trade names of Isopar or Permethyl, the C₈ to C₁₆ branched esters such as isohexyl or isodecyl neopentanoate, alcohols, and their mixtures. Preferably, the volatile hydrocarbon-based oils have a flash point of at least 40° C.

Examples of volatile hydrocarbon-based oils include, but are not limited to those given in Table 1 below.

TABLE 1

| Compound | Flash Point (° C.) |
|---|---|
| Isododecane | 43 |
| Isohexadecane | 102 |
| Isodecyl neopentanoate | 118 |
| Propylene glycol n-butyl ether | 60 |
| Ethyl 3-ethoxypropionate | 58 |
| Propylene glycol methylether acetate | 46 |
| Isopar L (isoparaffin C11-C13) | 62 |
| Isopar H (isoparaffin C11-C12) | 56 |

According to another embodiment, the composition may contain one or more non-volatile hydrocarbon-based oils, which may be of natural and synthetic origin. Examples of suitable non-volatile hydrocarbon-based oils which may be used in the compositions of the present invention include, but are not limited to,:
plant oils with a high triglyceride content consisting of fatty acid esters of glycerol, the fatty acids of which may have varied chain lengths, these chains possibly being linear or branched, and saturated or unsaturated; these oils are especially wheat germ oil, corn oil, sunflower oil, karite butter, castor oil, sweet almond oil, macadamia oil, apricot oil, soybean oil, rapeseed oil, cottonseed oil, alfalfa oil, poppy oil, pumpkin oil, sesame seed oil, marrow oil, avocado oil, hazelnut oil, grape seed oil, blackcurrant seed oil, evening primrose oil, millet oil, barley oil, quinoa oil, olive oil, rye oil, safflower oil, candlenut oil, passion flower oil or musk rose oil; or caprylic/capric acid triglycerides, for instance those sold by the company Stéarineries Dubois or those sold under the names Miglyol 810, 812 and 818 by the company Dynamit Nobel;
synthetic oils or esters of formula R₅COOR₆ in which R₅ represents a linear or branched higher fatty acid residue containing from 1 to 40 carbon atoms, including from 7 to 19 carbon atoms, and R₆ represents a branched hydrocarbon-based chain containing from 1 to 40 carbon atoms, including from 3 to 20 carbon atoms, with R₆+R₇≧10, such as, for example, Purcellin oil (cetostearyl octanoate), isononyl isononanoate, C₁₂ to C₁₅ alkyl benzoate, isopropyl myristate, 2-ethylhexyl palmitate, and octanoates, decanoates or ricinoleates of alcohols or of polyalcohols; hydroxylated esters, for instance isostearyl lactate or diisostearyl malate; and pentaerythritol esters;
synthetic ethers containing from 10 to 40 carbon atoms;
C₈ to C₂₆ fatty alcohols, for instance oleyl alcohol;
liquid paraffins and derivatives thereof, liquid petrolatum, liquid lanolin, polydecenes, hydrogenated polyisobutene such as Parleam®, squalane; and
mixtures thereof.

The composition of the present invention may also contain one or more volatile silicone oils, which may be linear or cyclic, having a viscosity, at room temperature, of less than or equal to 6 cSt, and having from 2 to 7 silicon atoms, optionally substituted with alkyl or alkoxy groups of 1 to 10 carbon atoms.

Examples of suitable volatile silicone oils include, but are not limited to, those listed in Table 2 below.

TABLE 2

| | Flash Point | Viscosity |
|---|---|---|
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane (L4) | 63 | 1.7 |
| KF-96 A from Shin Etsu | 94 | 6 |
| PDMS (polydimethylsiloxane) DC 200 | 56 | 1.5 |
| (1.5 cSt) from Dow Corning | | |
| PDMS DC 200 (2 cSt) from Dow Corning | 87 | 2 |
| PDMS DC 200 (5 cSt) from Dow Corning | 134 | 5 |
| PDMS DC 200 (3 St) from Dow Corning | 102 | 3 |

Other examples of volatile silicone oils include, but are not limited to, linear silicone oils such as those described in U.S. Pat. No. 6,338,839 and WO03/042221, the entire contents of which are incorporated herein by reference.

The composition of the present invention may also contain one or more non-volatile silicone oils. Examples of suitable non-volatile silicone oils include, but are not limited to, polydialkylsiloxanes, such as polydimethylsiloxanes (PDMS); polydimethylsiloxanes having alkyl, alkoxy or phenyl groups, which are pendent or at the end of a silicone chain; phenyl silicones, such as phenyl trimethicones, phenyl dimethicones, phenyl trimethylsiloxy diphenylsiloxanes, diphenyl dimethicones, diphenyl methyldiphenyltrisiloxanes and polymethylphenylsiloxanes; polysiloxanes modified with fatty acids (especially of C₈-C₂₀), fatty alcohols (especially of C₈-C₂₀) or polyoxyalkylenes; amino polysiloxanes; polysiloxanes containing hydroxyl groups; fluoro polysiloxanes comprising a fluorinated group that is pendent or at the end of a silicone chain, containing from 1 to 12 carbon atoms, all or some of the hydrogen atoms of which are replaced with fluorine atoms; and mixtures thereof.

In one embodiment, the compositions of the present invention are substantially free of silicone oils (i.e., contain less than about 5% silicone oils). In another embodiment, the compositions are substantially free of hydrocarbon-based oils (i.e., contain less than about 5% hydrocarbon-based oils). In another embodiment, the compositions are substantially free of non-volatile oils (i.e., contain less than about 5% non-volatile oils).

The oil phase is present in the composition in an amount of from about 1 to about 99% by weight; such as from about 5 to about 95% by weight; such as from about 10 to about 80% by weight; such as from about 15 to about 60% by weight; such as from about 20 to about 50% by weight, all weights being based on the total weight of the composition.

Cured Silicone Powder Coated with Microfine Particles

The cured silicone powder coated with microfine particles used in the present invention is described in U.S. Pat. No. 5,492,945, U.S. Pat. No. 5,756,568 and U.S. Pat. No. 5,945,471, the entire contents of which are hereby incorporated by reference.

The cured silicone powder is a silicone powder with an average (number average) particle size of from about 0.1 to about 500 μm; such as from about 0.5 to about 200 μm; such as from about 0.5 to about 20 μm. The nature of the cured silicone may be, for example, a gel, rubber, or cured resin, with rubbers being preferred. The cured silicone powder may have, for example, a spherical, disk-shaped, amorphous shape. A spherical shape is preferred.

Suitable microfine particles which can be used to coat the cured silicone powder include, but are not limited to, inorganic particles such as silicon oxide (silica), titanium oxide, aluminum oxide, zirconium oxide, antimony oxide, and other metal oxide microfine inorganic particles; boron nitride, aluminum nitride, and other metal nitride microfine particles; sulfate microfine particles, chloride microfine particles, and the like. Preferred microfine particles are metal oxide microfine particles, with silica microfine particles being most preferred. Also suitable are hydrophobicized microfine particles obtained by subjecting the surface of the microfine particles to hydrophobic treatment with organoalkoxysilane, organochlorosilane, organosilazane and other organosilicon compounds.

The microfine particles have a size sufficient for coating the surface of the cured silicone powder. A preferred particle size should be less than 1/10 of the average particle size of the cured silicone powder. Moreover, the specific surface area of the microfine particles should be greater than 10 m²/g.

A preferred cured silicone powder coated with microfine inorganic particles is a spherical silicone elastomer powder coated with silica, commercially available from Dow Corning under the tradename DC9701.

The cured silicone powder coated with microfine particles is present in an amount of from about 0.1 to about 90% by weight; such as from about 0.2 to about 60% by weight; such as from about 0.5 to about 40% by weight; such as from about 1 to about 20% by weight; such as from about 1 to about 10% by weight; such as from about 1 to about 5% by weight, all weights being based on the total weight of the composition.

Additives/Auxiliaries

The composition of the invention may also comprise any additive or auxiliary typically used in the field of cosmetic formulation. Examples thereof include, but are not limited to, dispersants such as poly(2-hydroxystearic acid), antioxidants, essential oils, preserving agents, humectants, fragrances, waxes, liposoluble polymers that are dispersible in the medium, fillers, neutralizing agents, cosmetic and dermatological active agents such as, for example, emollients, moisturizers, vitamins, essential fatty acids, sunscreens, colorants and mixtures thereof.

The composition of the invention may also contain, as an additive, an aqueous phase containing water that is optionally thickened or gelled with a thickener or gelling agent. The water may be present in an amount of from about 0.5 to about 50% by weight; such as from about 0.5 to about 30% by weight, based on the total weight of the composition. It should be noted, however, that anhydrous compositions are preferred. Particularly preferred are anhydrous compositions in the form of sticks or compacts. By “anhydrous composition”, it is meant that the composition contains less than 0.5% added water.

Suitable waxes are those generally used in cosmetics and dermatology. They include, for example, waxes of natural origin, such as beeswax, carnauba wax, candelilla wax, ouricury wax, Japan wax, cork fiber wax, sugar cane wax, paraffin wax, lignite wax, microcrystalline waxes, lanolin wax, montan wax, ozokerites and hydrogenated oils such as hydrogenated jojoba oil, as well as waxes of synthetic origin, for instance polyethylene waxes derived from the polymerization of ethylene, waxes obtained by Fischer-Tropsch synthesis, fatty acid esters and glycerides that are solid at 40° C. and above, silicone waxes such as alkyl- and alkoxy-poly(di)methylsiloxanes and/or poly(di)methylsiloxane esters that are solid at 40° C. and above. Preferred waxes that may be used in the composition of the invention include polyethylene and silicone waxes, preferably having a number average molecular weight of approximately 400-1500, but higher and lower molecular weights can be used.

Additional ingredients which may be used in the composition include crystalline silicone compounds. A crystalline silicone compound is a compound comprising silicone in its molecule, which is solid at room temperature, and has a crystalline character. Particularly useful crystalline silicone compounds belong to a class of alkyl siloxane waxes corresponding to the formulae below:
[(CH₃)₃SiO]₂(CH₃)SiR

(CH₃)₃SiO[(CH₃)₂SiO](RCH₃SiO)_{y}Si(CH₃)₃, which could also be written as R₃SiO[(CH₃)₂SiO]ₓ(RCH₃SiO)_{y}SiR₃,

where R is an alkyl chain, x may be 0-200 and y may be 0-200. The substituent R denotes an alkyl chain that may be as low as 1 or as high as 50 or more carbons as long as this silicone compound crystallizes at room temperature. Examples of crystalline silicone compounds include, but are not limited to, C₂₀-C₂₄ Alkyl Methicone, C₂₄-C₂₈ Alkyl Methicone, C₂₀-C₂₄ Alkyl Dimethicone, C₂₄-C₂₈ Alkyl Dimethicone commercially available from Archimica Fine Chemicals, Gainesville, Fla. under the designation of SilCare 41M40, SilCare 41M50, SilCare 41M70 and SilCare 41M80, C₃₀-C₄₅ Alkyl Methicone available from Dow Corning as AMS-C₃₀ Wax, as well as from General Electric as SF1642, or SF-1632. Other examples of crystalline silicone compounds include, but are not limited to, Stearyl Dimethicone available as SilCare 41M65 from Archimica or as DC-2503 from Dow-Corning, Midland, Mich., Stearoxytrimethylsilane sold as SilCare 1M71 or DC-580. Yet other examples of crystalline silicone compounds include, but are not limited to those available from Degussa Care Specialties, Hopewell, Va. under the designation ABIL Wax 9810, 9800, or 2440, or from Wacker-Chemie GmbH, Burghausen, Germany, under the designation BelSil SDM 5055, or from OSi Specialties, Greenwich, Conn. under the designation Silsoft.

Film-Forming Polymers

The compositions according to the invention may also comprise at least one film-forming polymer. Examples of suitable film-forming polymers include, but are not limited to, (meth)acrylates and their derivatives; vinylpyrrolidone (VP) copolymers and especially copolymers of vinylpyrrolidone and of a C₂-C₄₀ and in particular C₃-C₂₀ alkene and their derivatives; acrylic-silicone grafted polymers and their derivatives; fluoro polymers and their derivatives; block copolymers containing ethylenic monomers, particularly styrene and their derivatives; cellulose and their derivatives; silicone resins and their derivatives, preferably those chosen from trimethylsiloxysilicates and polyalkyl(aryl)silsesquioxanes; polyurethanes and their derivatives; polyesters and their derivatives; polyacrylamides and their derivatives; water-soluble resins and their derivatives, sugars and their derivatives; alginates; chitosan; gum arabic; and mixtures thereof.

The film-forming polymers may be present in the composition in an amount of from about 0.1 to about 25% by weight; such as from about 1 to about 20% by weight; such as from about 1 to about 10% by weight, all weights based on the total weight of the composition.

Coloring Agents

The composition of the invention may further comprise at least one coloring agent (colorant). The coloring agent may be chosen from the lipophilic dyes, hydrophilic dyes, pigments and nacreous pigments (i.e., nacres) typically used in cosmetic or dermatological compositions, and mixtures thereof. Suitable lipophilic dyes include, but are not limited to, Sudan Red, D&C Red 17, D&C Green 6, β-carotene, soybean oil, Sudan Brown, D&C Yellow 11, D&C Violet 2, D&C Orange 5, quinoline yellow, annatto and their mixtures. Suitable hydrophilic dyes include, but are not limited to, beetroot juice or methylene blue.

The pigments may be white or colored, goniochromatic or not, mineral and/or organic, and coated or uncoated. Suitable mineral pigments for use in the composition of the invention include, but are not limited to, titanium dioxide, zirconium oxide, zinc oxide, cerium oxide, iron oxide, chromium oxide, which are optionally surface-treated, manganese violet, ultramarine blue, chromium hydrate and ferric blue. Suitable organic pigments include, but are not limited to, carbon black, pigments of D & C type, and lakes based on cochineal carmine or on barium, strontium, calcium or aluminum.

The nacreous pigments may be chosen from white or colored nacreous pigments. Suitable nacreous pigments include, but are not limited to mica coated with titanium or with bismuth oxychloride, titanium mica with iron oxides, titanium mica with ferric blue or chromium oxide, titanium mica with an organic pigment.

Other suitable pigments may include interference pigments or those pigments having goniochromatic or flop effects.

In one embodiment, the coloring agent is a pigment (nacreous or not).

The coloring agents can be present in an amount of from about 0.01 to about 50% by weight; such as of from about 0.5 to about 40% by weight, more preferably of from about 2 to about 30% by weight, all weights being based on the total weight of the composition.

Sunscreens

The composition according to the invention may comprise a sunscreen capable of screening out UV radiation. The sunscreen may consist of one or more organic screening agents and/or one or more mineral (nano)pigments.

Suitable organic sunscreens include, but are not limited to, anthranilates, cinnamic derivatives, dibenzoylmethane derivatives, salicylic derivatives, camphor derivatives, triazine derivatives, benzophenone derivatives, β,β-diphenylacrylate derivatives, benzotriazole derivatives, benzalmalonate derivatives, benzimidazole derivatives, imidazolines, bis-benzazolyl derivatives, p-aminobenzoic acid (PABA) derivatives, methylenebis(hydroxyphenylbenzotriazole) derivatives, screening polymers and screening silicones, dimers derived from α-alkylstyrene, 4,4-diarylbutadienes, and mixtures thereof.

Suitable mineral sunscreens include, but are not limited to, pigments or even nanopigments (mean size of the primary particles: generally between 5 nm and 100 nm and preferably between 10 nm and 50 nm) of uncoated or coated metal oxides. Suitable nanopigments effective as UV photoprotective agents include, but are not limited to, oxide of titanium (amorphous or crystallized in rutile and/or anatase form), iron, zinc, zirconium, cerium and their mixtures. Suitable coating agents include, but are not limited to alumina and aluminum stearate.

The sunscreen may be present in the composition in an amount of from about 0.1 to about 30% by weight; such as from about 0.5 to about 20% by weight; such as from about 0.5 to about 15% by weight, based on the total weight of the composition.

The composition according to the invention may be manufactured by adapting known processes that are generally used in cosmetics or dermatology. It may be manufactured by a process which comprises mixing the necessary ingredients, optionally divided into phases. The mixture obtained can then be cast in a suitable mold such as a lipstick mold or directly into the packaging articles (case or dish in particular). Such processes are well within the ordinary skill of the artisan in view of this disclosure.

The composition according to the invention may, for example, be such that the composition is advantageously self-supporting and can disintegrate easily to form a satisfactory deposit on the keratinous substrates.

It should be noted that a person skilled in the art will take care to select the optional additional additives and/or the amount thereof such that the advantageous properties of the composition according to the invention are not, or are not substantially, adversely affected by the envisaged addition.

The composition according to the invention can be in any form including the form of a tinted or non tinted dermatological composition or a care composition for keratinous substrates, in the form of an antisun composition or body hygiene composition in particular in the form of a stick. It can be used in particular as a care base for the skin, hair, or the lips such as lip balms or care creams for the skin, the nails or the hair. It can be in the form of an emulsion, such as for example, a water in oil (W/O) emulsion, or an oil in water (O/W) emulsion, or a multiple emulsion, such as for example, W/O/W or O/W/O. The composition may also be in the form of a gel.

The composition of the invention may also be in the form of a colored make-up product for the skin, in particular a foundation, optionally having care or treating properties, a blusher, an eye shadow, a concealer product, an eyeliner, a make-up product for the body; a make-up product for the lips, optionally having care or treating properties; a make-up product for the nails or the eyelashes, in particular in the form of a mascara, or for the eyebrows and the hair, in particular in the form of a pencil.

### EXAMPLES

### Example 1

### Compact Foundation Formulation

| INCI NAME | Trade Name | % w/w |
|---|---|---|
| Isononyl Isononanoate | Wickenol 151 | 35.12 |
| Isohexadecane | Permethyl 101A | 15.00 |
| Nylon-611/Dimethicone Copolymer | DC2-8179 gellant | 10.25 |
| Dimethicone Crosspolymer/Silica | DC9701 Powder | 2.00 |
| Treated Pigments | | 10.26 |
| Titanium Dioxide | MT-100-Z | 7.52 |
| Fillers | | 19.85 |

This compact foundation exhibited heat stability and provided a unique texture and a cushiony skin feel.

### Example 2

### Compact Foundation Formulation

| INCI NAME | Trade Name | % w/w |
|---|---|---|
| Isononyl Isononanoate | Wickenol 151 | 34.97 |
| Isohexadecane | Permethyl 101A | 15.00 |
| Nylon-611/Dimethicone Copolymer | DC2-8179 gellant | 10.25 |
| Dimethicone Crosspolymer/Silica | DC9701 Powder | 4.00 |
| Treated Pigments | | 10.26 |
| Titanium Dioxide | MT-100-Z | 7.52 |
| Fillers | | 18.00 |

This compact foundation exhibited heat stability and provided a unique texture and a cushiony skin feel.

### Comparative Example 3

| INCI NAME | Trade Name | % w/w |
|---|---|---|
| Isononyl Isononanoate | Wickenol 151 | 36.78 |
| Isohexadecane | Permethyl 101A | 15.00 |
| Nylon-611/Dimethicone Copolymer | DC2-8179 gellant | 10.25 |
| Dibutyl lauroyl glutamide | GP-1 | 0.14 |
| Treated Pigments | | 10.26 |
| Titanium Dioxide | MT-100-Z | 7.52 |
| Fillers | | 19.85 |
| Simethicone | | 0.20 |

This comparative example lacked the cushiony, soft, smooth powdery feel.

### Examples 4 and 5

### Lip Gloss Formulations in a Pot

| | | | Compar- | Inven- |
|---|---|---|---|---|
| | | | ative | tive |
| | | TRADE | Ex. 4 | Ex. 5 |
| PHASE | INCI NAME | NAME | % w/w | % w/w |
| A | Nylon-611/Dimethicone | DC2-8179 | 15.0 | 15.0 |
| | Copolymer | gellant | | |
| | Isononyl Isononanoate | Wickenol | 26.4 | 21.4 |
| | | 151 | | |
| | Octyldodecyl Neopentanoate | Elefac | 11.0 | 11.0 |
| | | I-205 | | |
| | Phenyltrimethicone | | 28.6 | 28.6 |
| | (20 cst) | | | |
| | Trimethyl Pentaphenyl | DC 555 | 6.0 | 6.0 |
| | Trisiloxane & Tetramethyl | | | |
| | Hexaphenyl Tetrasiloxane | | | |
| | Bis-hydroxyethoxypropyl | DC5562 | 10.0 | 10.0 |
| | Dimethicone | Carbinol | | |
| | | fluid | | |
| B | Pigments | | 1.3 | 1.3 |
| | Diisopropyl Dimer | | 1.7 | 1.7 |
| | Dilinoleate | | | |
| C | Dimethicone | DC9701 | 0 | 5.0 |
| | Crosspolymer/Silica | powder | | |
| | TOTAL | | 100.0 | 100.0 |

The lip gloss in comparative Example 4 was grainy and lacked a cushiony texture.

The lip gloss in Example 5 was smooth and had a cushiony feel when applied onto the lips.

### Examples 6 and 7

### Mascara Formulations

| | | | Comparative | Inventiive |
|---|---|---|---|---|
| | | | Ex. 6 | Ex. 7 |
| PHASE | INCI NAME | TRADE NAME | % w/w | % w/w |
| A1 | Isododecane | PERMETHYL 99A | 20.45 | 16.45 |
| | Nylon-611/Dimethicone | DC2-8179 | 6.00 | 6.00 |
| | Copolymer | Gellant | | |
| A2 | Trimethylsiloxysilicate | SR-1000 | 9.00 | 9.00 |
| | Polyglyceryl-4 | ISOLAN GI 34 | 2.00 | 2.00 |
| | Isostearate | | | |
| | Cetyl PEG/PPG-10/1 | ABIL EM 90 | 0.50 | 0.50 |
| | Dimethicone | | | |
| | Bis-PEG-15 Methyl | BELSIL DMC | 0.80 | 0.80 |
| | Ether Dimethicone | 6038 | | |
| | Isododecane (60%) and | | 3.50 | 3.50 |
| | Acrylates/Dimethicone Copolymer | | | |
| | Black Iron Oxide | | 6.00 | 6.00 |
| | Beeswax | | 3.00 | 3.00 |
| A3 | Vinyl Dimethicone/Methicone | KSP-100 | 1.00 | 1.00 |
| | Silsesquioxane Crosspolymer | | | |
| A4 | Disteardimonium | BENTONE 38V | 2.20 | 2.20 |
| | Hectorite | | | |
| | Propylene Carbonate | | 0.95 | 0.95 |
| B | Water | | 40.25 | 40.25 |
| | Sodium Chloride | | 1.00 | 1.00 |
| | Pentylene Glycol | HYDROLITE-5 | 2.00 | 2.00 |
| | Sodium Dehydroacetate | | 0.20 | 0.20 |
| | Methylparaben | | 0.25 | 0.25 |
| | Laureth-4 | BRIJ 30 | 0.50 | 0.50 |
| | Phenoxyethanol | | 0.40 | 0.40 |
| | Dimethicone | DC 9701 | 0.00 | 4.00 |
| | Vinyldimethicone | cosmetic | | |
| | Crosspolymer | powder | | |
| | TOTAL | | 100.00 | 100.00 |

Product from inventive Example 7 was thicker, had more deposit, slip and cushiony texture and was less tacky than the comparative composition of Example 6.

### Examples 8 and 9

### Eye Shadow Formulations

| | | | Compar- | Inven- |
|---|---|---|---|---|
| | | | ative | tive |
| | | TRADE | Ex. 8 | Ex. 9 |
| PHASE | INCI NAME | NAME | % w/w | % w/w |
| A | Isododecane | PERMETH- | 13.0 | 11.0 |
| | | YL 99A | | |
| | Trimethylsiloxysilicate | SR-1000 | 4.5 | 4.5 |
| B | Iron Oxides | | 2.6 | 2.6 |
| | Isododecane | | 13.0 | 13.0 |
| | Nylon-611/Dimethicone | DC2-8179 | 3.0 | 3.0 |
| | Copolymer | Gellant | | |
| | Disteardimonium | | 32.2 | 32.2 |
| | Hectorite/Propylene | | | |
| | Carbonate in | | | |
| | Isododecane | | | |
| C | Silica | | 6.0 | 6.0 |
| | Talc | | 7.0 | 7.00 |
| | Mica | | 5.0 | 5.0 |
| | Mica and Iron Oxides | | 13.7 | 13.7 |
| | Dimethicone Vinyl | DC 9701 | 0.0 | 2.0 |
| | Dimethicone | Cosmetic | | |
| | Crosspolymer | powder | | |
| | TOTAL | | 100.0 | 100.0 |

Inventive example 9 had a better texture, and cushiony feel than comparative example 8.

What is claimed is:

1. A method of making-up a keratinous substrate comprising contacting the keratinous substrate with a composition comprising:
(a) at least one polyorganosiloxane-containing polymer having at least one moiety containing:
(i) from 1 to about 1000 organosiloxane units; and
(ii) at least two groups capable of establishing hydrogen interactions;
(b) an oil phase containing at least one oil component; and
(c) at least one cured silicone powder coated with microfine particles.

2. The method of claim 1, wherein (a) (ii) is chosen from an ester, an amide, a sulphonamide, a carbamate, a thiocarbamate, a urea, a thiourea, and combinations thereof.

3. The method of claim 1, wherein the polyorganosiloxane-containing polymer comprises at least one moiety corresponding to a formula chosen from formula (III): 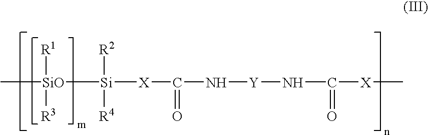
and formula (IV): 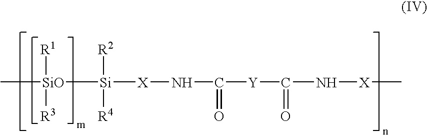
wherein R¹, R², R³ and R⁴ are the same or different and are chosen from the group consisting of methyl, ethyl, propyl, isopropyl, a siloxane chain, and phenyl; X is a linear or branched chain alkylene group having 1-30 carbons; Y is a linear or branched chain alkylene group having from 1 to about 40 carbons; m is an integer from 1 to about 700; and n is an integer from 1 to about 500.

4. The method of claim 1, wherein the at least one polyorganosiloxane-containing polymer is present in an amount of from about 0.1 to about 50% by weight, based on the total weight of the composition.

5. The method of claim 1, wherein the at least one polyorganosiloxane-containing polymer is present in an amount of from about 2 to about 30% by weight, based on the total weight of the composition.

6. The method of claim 1, wherein the oil phase contains at least one volatile hydrocarbon-based oil and at least one non-volatile hydrocarbon-based oil.

7. The method of claim 1, wherein the oil phase is present in an amount ranging from about 1 to about 99% by weight, based on the total weight of the composition.

8. The method of claim 1, wherein the oil phase is present in an amount ranging from about 15 to about 60% by weight, based on the total weight of the composition.

9. The method of claim 1, wherein the at least one cured silicone powder coated with microfine particles is a cured silicone powder coated with metal oxide microfine particles.

10. The method of claim 1, wherein the at least one cured silicone powder coated with microfine particles is a cured silicone powder coated with silica microfine particles.

11. The method of claim 1, wherein the cured silicone powder has an average particle size ranging from about 0.1 μm to about 200 μm.

12. The method of claim 1, wherein the cured silicone powder has an average particle size ranging from about 0.5 μm to about 25 μm.

13. The method of claim 1, wherein the microfine particles used to coat the cured silicone powder have an average particle size of less than about one-tenth of the average particle size of the cured silicone powder.

14. The method of claim 1, wherein the cured silicone powders coated with microfine particles are present in an amount of from about 0.1 to about 90% by weight, based on the total weight of the composition.

15. The method of claim 1, wherein the cured silicone powders coated with microfine silica are present in an amount of from about 0.5 to about 40% by weight, based on the total weight of the composition.

16. The method of claim 1, wherein the composition is a rigid gel.

17. The method of claim 1, wherein the composition is an emulsion.

18. The method of claim 1, wherein the composition is anhydrous.

19. The method of claim 1, wherein the composition is in a form chosen from a foundation, a blush, a concealer, a lipstick, a mascara, an eyeshadow and a sunscreen.
